(19) United States
(12) Patent Application Publication
(10) Pub. No.: US 2025/0359840 A1
(43) Pub. Date: Nov. 27, 2025

(54) DETECTION AND MITIGATION OF RADIATION EXPOSURE IN MEDICAL ENVIRONMENTS

(71) Applicant: Intuitive Surgical Operations, Inc., Sunnyvale, CA (US)

(72) Inventors: Omid Mohareri, San Francisco, CA (US); Muhammad Abdullah Jamal, Sunnyvale, CA (US); Reza Khodayi Mehr, Sunnyvale, CA (US)

(73) Assignee: Intuitive Surgical Operations, Inc., Sunnyvale, CA (US)

(21) Appl. No.: 19/203,838

(22) Filed: May 9, 2025

Related U.S. Application Data
(60) Provisional application No. 63/651,893, filed on May 24, 2024.

(51) Int. Cl. A61B 6/00; A61B 6/46; G01T 1/02; G06V 10/70; G06V 20/50; G06V 40/10

(52) U.S. Cl. CPC A61B 6/542; A61B 6/463; A61B 6/5229; G01T 1/02; G06V 10/70; G06V 20/50; G06V 40/10; G06V 2201/03

(57) ABSTRACT

Aspects of this technical solution can determine, by a first machine learning model based on a first input comprising data for a medical procedure performed in the medical environment, a first output identifying an object or a person at a first location in the medical environment, the data including one or more images captured by a sensor within the medical environment, determine, respective to a second location of a radiation-emitting device in a medical environment, one or more radiation metrics corresponding to propagation of radiation from the radiation-emitting device through the medical environment, and generate, by a second machine learning model based on a second input comprising the radiation metrics, the first location, and the first output, a second output indicative of a quantity of radiation exposure of the object or the person at the first location.

### CROSS-REFERENCE TO RELATED APPLICATION

This application claims the benefit of, and priority to, U.S. Patent Application No. 63/651,893, filed May 24, 2024, the full disclosure of which is incorporated herein in its entirety.

### TECHNICAL FIELD

The present implementations relate generally to medical devices, including but not limited to detection and mitigation of radiation exposure in medical environments.

### INTRODUCTION

Radiation can provide significant clinical benefits and can be an impactful tool for surgical and clinical procedures. However, benefits of controlled application of radiation are counteracted by risks of radiation exposure by those near radiation emitting devices. However, conventional systems cannot account for radiation at sufficient accuracy to be effective in managing exposure in highly sensitive environments.

### SUMMARY

Systems, methods, apparatuses, and non-transitory computer-readable media are provided for determining and/or modeling radiation exposure of people and/or objects in a medical environment. A system according to this technical solution can determine propagation and scattering of radiation emitted from a given source (e.g., X-ray emission), with respect to locations and characteristics of specific people and objects in the environment. This solution can identify quantitative values of radiation at one or more surfaces of one or more people within a physical space, based on an amount of radiation propagation over a given volume between the source of the radiation and the surface of the person or object, aggregated over time. Thus, a technical solution for detection and mitigation of radiation exposure in medical environments is provided.

At least one aspect is directed to a system. The system can include one or more processors, coupled with memory. The system can determine, by a first machine learning model based on a first input can include data for a medical procedure performed in the medical environment, a first output identifying an object or a person at a first location in the medical environment, the data can include one or more images captured by a sensor within the medical environment. The system can determine, respective to a second location of a radiation-emitting device in a medical environment, one or more radiation metrics corresponding to propagation of radiation from the radiation-emitting device through the medical environment. The system can generate, by a second machine learning model based on a second input can include the radiation metrics, the first location, and the first output, a second output indicative of a quantity of radiation exposure of the object or the person at the first location.

At least one aspect is directed to a method. The method can include determining, by a first machine learning model based on a first input can include data for a medical procedure performed in the medical environment, a first output identifying an object or a person at a first location in the medical environment, the data can include one or more images captured by a sensor within the medical environment. The method can include determining, respective to a second location of a radiation-emitting device in a medical environment, one or more radiation metrics corresponding to propagation of radiation from the radiation-emitting device through the medical environment. The method can include generating, by a second machine learning model based on a second input can include the radiation metrics, the first location, and the first output, a second output indicative of a quantity of radiation exposure of the object or the person at the first location.

At least one aspect is directed to a non-transitory computer readable medium can include one or more instructions stored thereon and executable by a processor. The processor can determine, via a first machine learning model based on a first input can include data for a medical procedure performed in the medical environment, a first output identifying an object or a person at a first location in the medical environment, the data can include one or more images captured by a sensor within the medical environment. The processor can determine, the processor and respective to a second location of a radiation-emitting device in a medical environment, one or more radiation metrics corresponding to propagation of radiation from the radiation-emitting device through the medical environment. The processor can generate, via a second machine learning model based on a second input can include the radiation metrics, the first location, and the first output, a second output indicative of a quantity of radiation exposure of the object or the person at the first location.

### BRIEF DESCRIPTION OF THE FIGURES

These and other aspects and features of the present implementations are depicted by way of example in the figures discussed herein. Present implementations can be directed to, but are not limited to, examples depicted in the figures discussed herein. Thus, this disclosure is not limited to any figure or portion thereof depicted or referenced herein, or any aspect described herein with respect to any figures depicted or referenced herein.

FIG. 1 depicts an example environment of a system according to this disclosure.

FIG. 2 depicts an example medical environment with radiation propagation, according to this disclosure.

FIG. 3 depicts an example system, according to this disclosure.

FIG. 4 depicts an example computer architecture, according to this disclosure.

FIG. 5 depicts an example layer model architecture, according to this disclosure.

FIG. 6A depicts an example detection of radiation surface characteristics, according to this disclosure.

FIG. 6B depicts an example detection of radiation exposure, according to this disclosure.

FIG. 6C depicts an example mitigation of radiation exposure, according to this disclosure.

FIG. 7 depicts an example method of detection and mitigation of radiation exposure in medical environments, according to this disclosure.

FIG. 8 depicts an example method of detection and mitigation of radiation exposure in medical environments, according to this disclosure.

### DETAILED DESCRIPTION

Aspects of this technical solution are described herein with reference to the figures, which are illustrative examples of this technical solution. The figures and examples below are not meant to limit the scope of this technical solution to the present implementations or to a single implementation, and other implementations in accordance with present implementations are possible, for example, by way of interchange of some or all of the described or illustrated elements. Where certain elements of the present implementations can be partially or fully implemented using known components, only those portions of such known components that are necessary for an understanding of the present implementations are described, and detailed descriptions of other portions of such known components are omitted to not obscure the present implementations. Terms in the specification and claims are to be ascribed no uncommon or special meaning unless explicitly set forth herein. Further, this technical solution and the present implementations encompass present and future known equivalents to the known components referred to herein by way of description, illustration, or example.

In certain aspects, intraoperative imaging devices (e.g., fluoroscopy) have significant clinical benefits and uses, and it is important to minimize risk of radiation exposure for the patient, surgeon, and surgical staff to prevent risk of harm. Traditional radiation exposure risk minimization methods rely on human observations, which is prone to error and bias, not to mention the exceptional high implementation cost and the lack of scalability and accuracy. No system current exists that can perform real-time analysis of radiation exposure for patient, surgeon, and surgical staff during a medical procedure, much less providing any notification or indication of the radiation exposure risk during the medical procedure in real time.

The embodiments described herein can model radiation at one or more surfaces in real time during a medical procedure in which an imaging device emitting radiation is used and determine radiation exposure on the surfaces (of one or more people or objects) that exceeds a given threshold. For example, the system can determine that a surface in a model of a medical environment during a medical procedure is experiencing or receiving exposure to radiation above a recommended level, and can provide an indication that a person or object associated with the surface (e.g., a front of a torso or a head of a medical staff member) is receiving the instantaneous radiation above the recommended level. For example, the system can determine that a surface in a model of a medical environment for a medical procedure has received exposure to radiation above a recommended level (as defined using a threshold value) over a given period of time, either during or subsequent to the medical procedure. For example, the system can provide an indication that a person or object associated with the surface (e.g., a front of a torso or a head of a medical staff member) received an aggregate radiation above the recommended level. The system can annotate one or more images or frames of video to highlight various objects and people in an image or a video, and can provide, for example, annotations to the video can include (e.g., a color-based overlay indicating a quantitative level of radiation exposure, or text or images indicative of or descriptive of instantaneous or aggregate radiation associated with a specific person or object, or any portion of the medical environment).

The system can identify, according to a machine learning model configured to detect image features, one or more characteristics of an object or a person in the medical environment, and can modify a value of radiation exposure according to the characteristic. For example, a machine learning model can determine that a person in a medical environment is wearing a protective vest, and can reduce a quantitative value of radiation exposure associated with the torso of the person wearing the vest by an amount that accounts for the radiation absorption or radiation reflectivity of the vest. In some examples, a machine learning model as discussed herein, can account for locations and characteristics of objects with respect to radiation propagation, to accurately model propagation and scattering patterns for a given medical environment based on the specific objects and people in that environment.

Accordingly, the embodiments described herein can compute propagation of radiation in a given medical environment and identify amounts of radiation on given objects or people in the given medical environment during a medical procedure. A coordinate for a source of radiation can be determined with respect to one or more sensors in the medical environment (e.g., identify coordinates in a common coordinate space for a location of a radiation source in the medical environment and one or more locations of one or more cameras in the medical environment). The system can generate a point cloud corresponding to surfaces of the medical environment. The system can then correlate one or more points of a point cloud identifying surfaces of the medical environment, with corresponding radiation metrics indicating radiation exposure at each of those points.

The system can segment one or more images from one or more sensors into one or more objects. The system can identify a pose of a person at one or more times, and can identify aa interaction based on one pose of one person, or a plurality of poses for a plurality of corresponding persons (e.g., including poses of multiple people within a predetermined distance of one another to determine a type of interaction). The system can associate one or more radiation metrics with one or more of the segments (e.g., objects or people), and can provide indications if those metrics exceed radiation exposure thresholds (e.g., thresholds that vary for people or objects). The system can identify metrics based on radiation exposure, including objective performance indicators (OPIs) that correspond to various objects (e.g., a robotic system) or people (e.g., a surgeon or medical staff) in the medical environment, or the medical environment, or portions of the medical environment (e.g., an imaging area, an observation area). OPIs can include aggregate radiation exposure to one or more objects over the course of a given medical procedure, a phase of a given medical procedure, or a task of a phase. In another aspect, the system can be used to provide indications of aggregate radiation exposure of a particular person across multiple medical procedures performed in one or more medical environments over a time period (e.g., a day, a week, a month). In yet another aspect, the system can be used to provide medical environment recommendations to reduce or optimize radiation exposure of personnel within a particular medical environment.

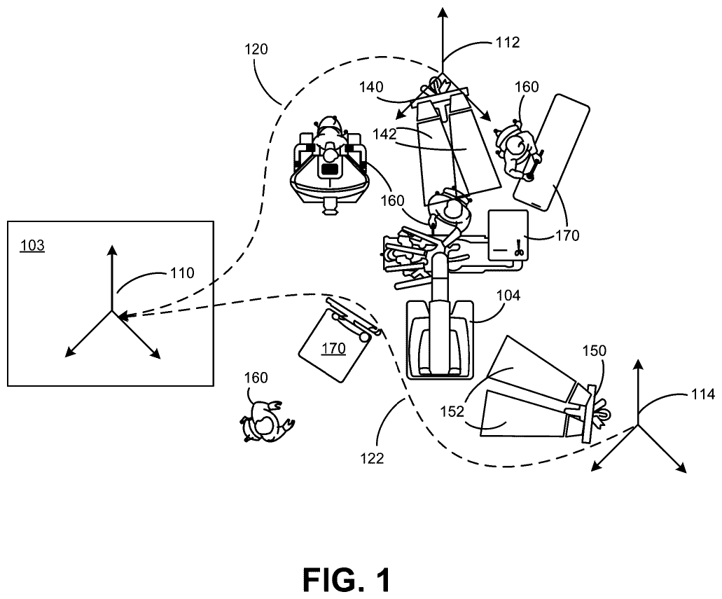

FIG. 1 depicts an example environment of a system according to this disclosure. As illustrated by way of example in FIG. 1, an environment 100 of a system 100 can include at least a radiation system 103, a robotic system 104, a first sensor system 140, a second sensor system 150, persons 160, and objects 170, and can operate according to at least a first coordinate frame 110, a second coordinate frame 112, a third coordinate frame 114, a first coordinate registration 120, and a second coordinate registration 122. For example, the environment 100 is illustrated by way of example as a plan view of an OR having the radiation system 103, the robotic system 104, the first sensor system 140, the second sensor system 150, the persons 160, and the objects 170 disposed therein or thereabout. This technical solution is not limited to a medical environment or an environment that includes or involves a robotic manipulator system as depicted herein by way of example, or any robotic system or surgical robotic system.

The radiation system 103 can include one or more radiation-emitting devices to provide radiation to a given location at a given level. For example, the radiation system 103 can correspond to a medical imaging system configured to generate one or more representations of a patient or a patient site based on electromagnetic radiation transmitted from the radiation system 103 to the patient or the patient site. For example, the radiation system 103 can generate a representation of at least one aspect of the patient or the patient site based on detection of radiation reflected or absorbed at the patient site. For example, the radiation system 103 can correspond to a radiotherapy system configured to apply electromagnetic radiation transmitted from the radiation system 103 to the patient or the patient site according to a medical treatment. For example, the radiation system 103 can generate apply radiation to the patient or the patient site at one or more levels at one or more times according to a radiotherapy treatment. The radiation system 103 can propagate radiation beyond a target location, area or volume associated with the patient or the patient site, and the propagation of radiation can be detectable at one or more locations, areas, or volumes extending or entirely beyond the target location, area or volume.

The robotic system 104 can include one or more robotic devices configured to perform one or more actions of a medical procedure (e.g., a surgical procedure). For example, a robotic device can include, but is not limited to a surgical device that can be manipulated by robotic device. For example, a surgical device can include, but is not limited to, a scalpel or a cauterizing tool. The robotic system 104 can include various motors, actuators, or electronic devices whose position or configuration can be modified according to input at one or more robotic interfaces. For example, a robotic interface can include a manipulator with one or more levers, buttons, or grasping controls that can be manipulated by pressure or gestures from one or more hands, arms, fingers, or feet. The robotic system 104 can include a surgeon console in which the surgeon can be positioned (e.g., standing or seated) to operate the robotic system 104. However, the robotic system 104 is not limited to a surgeon console co-located or on-site with the robotic system 104.

The presence, placement, orientation, and configuration, for example, of one or more of the robotic system 104, the first sensor system 140, the second sensor system 150, the persons 160, and the objects 170 can correspond to a given medical procedure or given type of medical procedure that is being performed, is to be performed, or can be performed in the OR corresponding to the environment 100. This disclosure is not limited to the presence, placement, orientation, or configuration of the robotic system 104, the first sensor system 140, the second sensor system 150, the persons 160, the objects 170, or any other element illustrated herein by way of example. For example, one or more cameras located at the robotic manipulator system 104 can capture a view of the surgical site via the sensor at or proximate to the robotic manipulator system 130 from outside the surgical site (e.g., above the surgical site and framing hands and tools of one or more surgeons and one or more anatomical features being operated on by the one or more surgeons). Thus, the robotic system 104 can capture a field of view from the one or more cameras that corresponds to a physical volume within the environment 100 that is within the range of detection of one or more sensors of the robotic system 104.

The first sensor system 140 can include one or more sensors oriented to a first portion of the environment 100. For example, the first sensor system 140 can include one or more cameras configured to capture images or video in visual or near-visual spectra and/or one or more depth-acquiring sensors for capturing depth data (e.g., three-dimensional point cloud data). For example, the first sensor system 140 can include a plurality of cameras configured to collectively capture images or video in a stereoscopic view. For example, the first sensor system 140 can include a plurality of cameras configured to collectively capture images or video in a panoramic view. The first sensor system 140 can include a field of view 142. The field of view 142 can correspond to a physical volume within the environment 100 that is within the range of detection of one or more sensors of the first sensor system 140. For example, the field of view 142 is oriented toward a surgical site of a patient. For example, the field of view 142 is located behind a surgeon at the surgical site of a patient.

The second sensor system 150 can include one or more sensors oriented to a second portion of the environment 100. For example, the second sensor system 150 can include one or more cameras configured to capture images or video in visual or near-visual spectra and/or one or more depth-acquiring sensors for capturing depth data (e.g., three-dimensional point cloud data). For example, the second sensor system 150 can include a plurality of cameras configured to collectively capture images or video in a stereoscopic view. For example, the second sensor system 150 can include a plurality of cameras configured to collectively capture images or video in a panoramic view. The second sensor system 150 can include a field of view 152. The field of view 152 can correspond to a physical volume within the environment 100 that is within the range of detection of one or more sensors of the second sensor system 150. For example, the field of view 152 is oriented toward the robotic system 104. For example, the field of view 152 is located adjacent to the robotic system 104.

The persons 160 can include one or more individuals present in the environment 100. For example, the persons 160 can include, but are not limited to, assisting surgeons, supervising surgeons, specialists, nurses, or any combination thereof. The objects 170 can include, but are not limited to, one or more pieces of furniture, instruments, or any combination thereof. For example, the objects 170 can include tables and surgical instruments.

The first coordinate frame 110 can correspond to the radiation system 103, and can define a first coordinate space relative to the radiation system 103. For example, the first coordinate space can correspond to a Cartesian coordinate space having a first origin at the radiation system 103 or defined relative to the radiation system 103. For example, the first origin can correspond to a center of the radiation system 103, a centroid of the radiation system 103, or a point of emission of radiation from the radiation system 103, but is not limited thereto. The second coordinate frame 112 can correspond to the first sensor system 140, and can define a second coordinate space relative to the first sensor system 140. For example, the second coordinate space can correspond to a Cartesian coordinate space having a second origin at the first sensor system 140 or defined relative to the first sensor system 140. For example, the second origin can correspond to a center of the first sensor system 140, a centroid of the first sensor system 140, or a view of a camera or a focal point of a camera of the first sensor system 140, but is not limited thereto. The third coordinate frame 114 can correspond to the second sensor system 150, and can define a third coordinate space relative to the second sensor system 150. For example, the third coordinate space can correspond to a Cartesian coordinate space having a third origin at the second sensor system 150 or defined relative to the second sensor system 150. For example, the third origin can correspond to a center of the second sensor system 150, a centroid of the second sensor system 150, or a view of a camera or a focal point of a camera of the second sensor system 150, but is not limited thereto. In an aspect, coordinates (e.g., Cartesian coordinates) of one or more coordinate spaces (e.g., a common coordinate space) can be determined by triangulation based on depth data from a plurality of sensors each registered to the common coordinate space.

The first coordinate registration 120 can correspond to a transformation of the second coordinate space to the first coordinate space. For example, the first coordinate registration 120 can be indicative of a transformation of coordinate tracking of the first sensor system 140 according to the first coordinate space and the first origin. For example, the first sensor system 140 can determine position and orientation in the environment 100 according to the first coordinate space and relative to the first origin. For example, the first coordinate registration 120 can be indicative of a translation of coordinate tracking of the first sensor system 140 according to the first coordinate space and the first origin. For example, the first sensor system 140 can determine position and orientation in the environment 100 according to the second coordinate space and relative to the second origin. For example, the first sensor system 140 or the data processing system 102 can translate one or more coordinates from the second coordinate space to the first coordinate space according to a coordinate offset indicating a difference in one or more coordinates between the first origin and the second origin. For example, a data processing system as discussed herein can translate the coordinates from the second coordinate space to the first coordinate space in substantially real time to provide a technical improvement to track radiation exposure in real time in the environment 100.

The second coordinate registration 122 can correspond to a transformation of the third coordinate space to the first coordinate space. For example, the first coordinate registration 120 can be indicative of a transformation of coordinate tracking of the second sensor system 150 according to the first coordinate space and the first origin. For example, the second sensor system 150 can determine position and orientation in the environment 100 according to the first coordinate space and relative to the first origin. For example, the first coordinate registration 120 can be indicative of a translation of coordinate tracking of the second sensor system 150 according to the first coordinate space and the first origin. For example, the second sensor system 150 can determine position and orientation in the environment 100 according to the third coordinate space and relative to the third origin. For example, the second sensor system 150 or the data processing system 102 can translate one or more coordinates from the third coordinate space to the first coordinate space according to a coordinate offset indicating a difference in one or more coordinates between the first origin and the third origin. For example, the data processing system 102 can translate the coordinates from the third coordinate space to the first coordinate space in substantially real time to provide a technical improvement to track radiation exposure in real time in the environment 100.

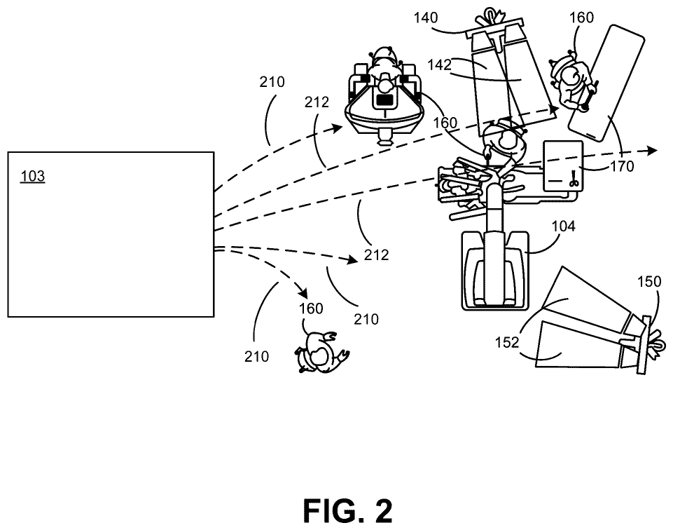

FIG. 2 depicts an example medical environment with radiation propagation, according to this disclosure. As illustrated by way of example in FIG. 2, a medical environment 200 with radiation propagation can include at least unobstructed paths of radiation propagation 210, and obstructed paths of radiation propagation 212. For example, the environment 200 can correspond to the environment 100, during a time or time period of activation of the radiation system 103. The unobstructed paths of radiation propagation 210 can correspond to unobstructed propagation of radiation from the radiation system 103 through the environment 200 to one or more persons 160 or objects 170. For example, an obstruction can correspond to a person 160 or an object 170 disposed at least partially between another person 160 or object 170, to at least partially absorb or reflect at least a portion of radiation emitted from the radiation system 103. Thus, the unobstructed paths of radiation propagation 210 are free of intervening persons 160 or objects 170, or can correspond to radiation exposure to those intervening persons 160 or objects 170. The obstructed paths of radiation propagation 212 can correspond to obstructed propagation of radiation from the radiation system 103 through the environment 200 to one or more persons 160 or objects 170, across one or more intervening persons 160 or objects 170.

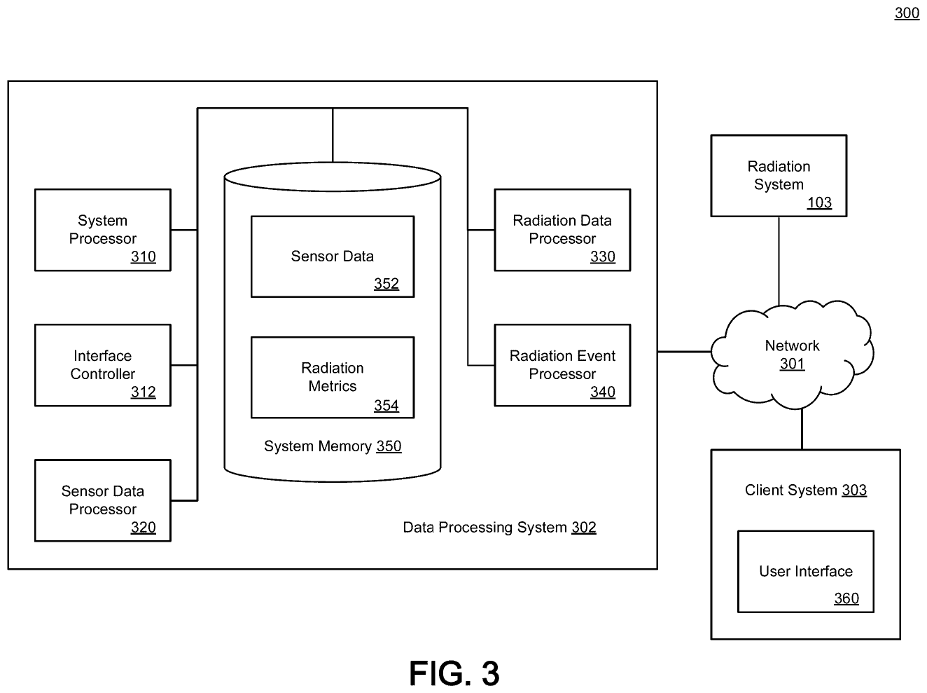

FIG. 3 depicts an example system, according to this disclosure. As illustrated by way of example in FIG. 3, a system 300 can include at least a network 301, a data processing system 302, a client system 303, and the radiation system 103.

The data processing system 302 can include a physical computer system operatively coupled or configured to couple with one or more components of the system 300, either directly or directly through an intermediate computing device or system. The data processing system 302 can include a virtual computing system, an operating system, and a communication bus to effect communication and processing. The data processing system 302 can include a system processor 310, an interface controller 312, a sensor data processor 320, a radiation data processor 330, a radiation event processor 340, and a system memory 350.

The network 301 can include any type or form of network. The geographical scope of the network 301 can vary widely and the network 301 can include a body area network (BAN), a personal area network (PAN), a local-area network (LAN), e.g., Intranet, a metropolitan area network (MAN), a wide area network (WAN), or the Internet. The topology of the network 301 can be of any form and can include, e.g., any of the following: point-to-point, bus, star, ring, mesh, or tree. The network 301 can include an overlay network which is virtual and sits on top of one or more layers of other networks 101. The network 301 can be of any such network topology as known to those ordinarily skilled in the art capable of supporting the operations described herein. The network 301 can utilize different techniques and layers or stacks of protocols, including, e.g., the Ethernet protocol, the Internet protocol suite (TCP/IP), the ATM (A synchronous Transfer Mode) technique, the SONET (Synchronous Optical Networking) protocol, or the SD (Synchronous Digital Hierarchy) protocol. The ‘TCP/IP Internet protocol suite can include application layer, transport layer, Internet layer (including, e.g., IPV6), or the link layer. The network 301 can include a type of a broadcast network, a telecommunications network, a data communication network, or a computer network.

The system processor 310 can execute one or more instructions associated with the system 300. The system processor 310 can include an electronic processor, an integrated circuit, or the like including one or more of digital logic, analog logic, digital sensors, analog sensors, communication buses, volatile memory, nonvolatile memory, and the like. The system processor 310 can include, but is not limited to, at least one microcontroller unit (MCU), microprocessor unit (MPU), central processing unit (CPU), graphics processing unit (GPU), physics processing unit (PPU), embedded controller (EC), or the like. The system processor 310 can include a memory operable to store or storing one or more instructions for operating components of the system processor 310 and operating components operably coupled to the system processor 310. For example, the one or more instructions can include one or more of firmware, software, hardware, operating systems, embedded operating systems. The system processor 310 or the system 300 generally can include one or more communication bus controller to effect communication between the system processor 310 and the other elements of the system 300.

The interface controller 312 can link the data processing system 102 with one or more of the network 301, the radiation system 103, the robotic system 104, and the client system 303, by one or more communication interfaces. A communication interface can include, for example, an application programming interface (“API”) compatible with a particular component of the data processing system 102, or the client system 303. The communication interface can provide a particular communication protocol compatible with a particular component of the data processing system 102 and a particular component of the client system 303. The interface controller 312 can be compatible with particular content objects and can be compatible with particular content delivery systems corresponding to particular content objects, structures of data, types of data, or any combination thereof. For example, the interface controller 312 can be compatible with transmission of text data or binary data structured according to one or more metrics or data of the system memory 350.

The sensor data processor 320 can identify one or more features in depictions in video data as discussed herein. For example, the depictions can include portions of a patient site, one or more medical instruments, or any combination thereof, but are not limited thereto. The sensor data processor 320 can identify one or more edges, regions, or a structure within an image and associated with the depictions. For example, an edge can correspond to a line in an image that separates two depicted objects (e.g., a delineation between a radiation vest and a limb or head of a person 160 wearing the vest). For example, a region can correspond to an area in an image that at least partially corresponds to a depicted object (e.g., a person 160 or a vest of the person 160). For example, a structure can correspond to an area in an image that at least partially corresponds to a portion of a depicted object or a predetermined type of an object (e.g., a scalpel edge). The sensor data processor 320 can receive and process data from one or more sensors (e.g., cameras) and can generate or transform data provided by one or more sensors to one or more formats compatible with image feature processing (e.g., converting RAW data into bitmap or vector image frames).

The radiation data processor 330 can generate one or more models to determine propagation of radiation in a given medical environment, and can determine one or more levels of radiation exposure at one or more surfaces of one or more persons 160 or objects 170 of the given medical environment. For example, the radiation data processor 330 can obtain one or more images or images features from the sensor data processor, and can determine radiation associated with one or more objects or portions of objects in the environment. For example, the radiation data processor 330 can determine propagation based on a coordinate system or one or more transformed or translated coordinate systems as discussed herein. Thus, the radiation data processor 330 can determine a level of radiation at a given time or aggregated over a plurality of times, with respect to one or more persons, objects, portions of persons, portions of objects, surfaces, portions of surfaces, or any combination thereof. In an aspect, the radiation data processor 330 can generate coordinates (e.g., Cartesian coordinates) of one or more coordinate spaces (e.g., a common coordinate space) by triangulating surface features based on depth data from a plurality of sensors each registered to the common coordinate space. For example, the radiation data processor 330 can triangulate portions of a surface based on depth data for a surface object identified as corresponding to the same portion of a volume by a 3D registration as discuss herein.

The radiation event processor 340 can determine radiation exposure at one or more portions of an environment, and can generate one or more outputs corresponding to the determined radiation exposure. For example, the radiation event processor 340 can determine radiation exposure at a time (e.g., instantaneous exposure) or at one or more times, time periods, or time ranges (e.g., aggregate exposure) with respect to one or more discrete portions of the environment. For example, a discrete portion of the environment can correspond to an individual person 160 or an individual object 170 in the environment 100 or 200. For example, a discrete portion of the environment can correspond to a portion of an individual person 160, including a discrete body part (e.g., head, limbs, torso) or a segmented portion of the person 160 (e.g., all portions of a person not covered by a protective vest). The radiation event processor 340 can determine the radiation exposure with respect to one or more surfaces of the portions of the environment, and can present or cause a user interface to present visual indications of the radiation exposure corresponding to the surfaces of the portions of the environment. For example, the radiation event processor 340 can provide one or more visual overlays having one or more colors corresponding to levels of radiation exposure, either instantaneous or aggregate. Thus, the radiation event processor 340 can provide a technical solution to provide radiation exposure determinations and feedback at a level of accuracy and responsiveness (e.g., real-time) beyond the capability of manual processes to achieve.

The system memory 350 can store data associated with the system 300. The system memory 350 can include one or more hardware memory devices to store binary data, digital data, or the like. The system memory 350 can include one or more electrical components, electronic components, programmable electronic components, reprogrammable electronic components, integrated circuits, semiconductor devices, flip flops, arithmetic units, or the like. The system memory 350 can include at least one of a non-volatile memory device, a solid-state memory device, a flash memory device, or a NAND memory device. The system memory 350 can include one or more addressable memory regions disposed on one or more physical memory arrays. A physical memory array can include a NAND gate array disposed on, for example, at least one of a particular semiconductor device, integrated circuit device, and printed circuit board device. The system memory 350 can include a sensor data 452, and radiation metrics 454.

The sensor data 452 can depict one or more medical procedures from one or more viewpoints associated with corresponding medical procedures. For example, the sensor data 452 can include video data that can correspond to still images or frames of video images that depict at least a portion of a medical procedure, medical environment, or patient site from a given viewpoint. For example, the sensor data 452 can include data associated with responses to stimulus by a light or electromagnetic sensor that can be converted to still images or frames of video images that depict at least a portion of a medical procedure, medical environment, or patient site from a given viewpoint. For example, the sensor data processor 320 can identify one or more depictions in an image or across a plurality of images. Each time can, for example, be associated with a given task or phase of a workflow as occurring during that task or phase.

The radiation metrics 454 can be indicative of radiation exposure thresholds corresponding to various persons 160, objects 170, or any combination thereof. For example, the radiation metrics 454 can be indicative of radiation exposure thresholds indicative of various levels of exposure (e.g., mitigated, within limit, exceeding limit, and mitigate now). For example, the radiation metrics 454 can be indicative of radiation exposure thresholds for various types of persons 160 (e.g., patient, medical staff, radiation system operator). For example, the radiation metrics 454 can be indicative of radiation metrics (e.g., absorptiveness or reflectivity) for various types of surfaces of (e.g., skin, face, clothing, protective vest, metal, plastic).

The client system 303 can include a computing system associated with a database system. For example, the client system 303 can correspond to a cloud system, a server, a distributed remote system, or any combination thereof. For example, the client system 303 can include an operating system to execute a virtual environment. The operating system can include hardware control instructions and program execution instructions. The operating system can include a high-level operating system, a server operating system, an embedded operating system, or a boot loader. The client system 303 can include a user interface 360. The user interface 360 can include one or more devices to receive input from a user or to provide output to a user. For example, the user interface 360 can correspond to a display device to provide visual output to a user and one or more or user input devices to receive input from a user. For example, the input devices can include a keyboard, mouse or touch-sensitive panel of the display device, but are not limited thereto. The display device can display at least one or more presentations as discussed herein, and can include an electronic display. An electronic display can include, for example, a liquid crystal display (LCD), a light-emitting diode (LED) display, an organic light-emitting diode (OLED) display, or the like. The display device can receive, for example, capacitive or resistive touch input. The display device can be housed at least partially within the client system 303.

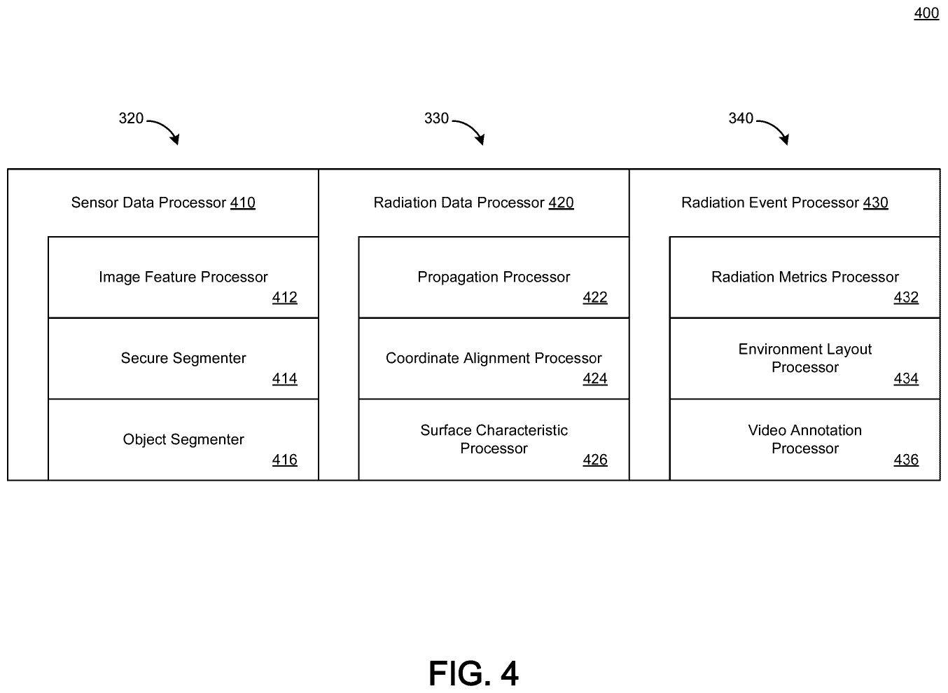

FIG. 4 depicts an example computer architecture, according to this disclosure. As illustrated by way of example in FIG. 4, a computer architecture 400 can include at least a sensor data processor 410, a radiation data processor 420, and a radiation event processor 430. The sensor data processor 410 can correspond at least partially in one or more of structure and operation to the sensor data processor 320. The sensor data processor 410 can include an image feature processor 412, a scene segmenter 414, and an object segmenter 416. In an aspect, the sensor data processor 410 can generate, by the first machine learning model, the first location in real time during the medical procedure. For example, the first machine learning model is a machine learning model configured to detect image features or identify persons or objects in an image or video, or any combination thereof. The image feature processor 412 can identify one or more features in depictions in video data as discussed herein. For example, the depictions can include portions of a patient site, one or more medical instruments, or any combination thereof, but are not limited thereto. The scene segmenter 414 can identify one or more persons 160 or objects 170 in one or more images according to the first machine learning model. For example, the first machine learning model can perform a scene segmentation to identify one or more shapes or edges between shapes that distinguish one or more persons, objects and environments from each other within an image or a video.

The object segmenter 416 can identify portions of one or more persons 160 or objects 170 in one or more images according to the first machine learning model. For example, the object segmenter 416 can identify one or more edges, regions, or a structure within an image and associated with the depictions. For example, the image feature processor 412 can identify a person, a vest worn by the person, and surface features of the person (e.g., skin, face, clothing portions). In an aspect, the object segmenter 416 can identify, by the first machine learning model, at least one item associated with the portion of the object or the portion of the person. The system can determine, by the second machine learning model based on the one or more items, the characteristic metric based on an association of the portion of the object or the portion of the person with the item. In an aspect, the portion of the object or the portion of the person corresponds to a portion of a point cloud associated with the object or the person. For example, the second machine learning model can be trained according to image data and radiation exposure metrics to identify absorptiveness or reflectivity of one or more types of surfaces of one or more persons, objects, or environments.

The radiation data processor 420 can correspond at least partially in one or more of structure and operation to the radiation data processor 330. The radiation data processor 420 can include a propagation processor 422, a coordinate alignment processor 424, and a surface characteristic processor 426. The propagation processor 422 can determine a quantity of radiation exposure at a given point in a volume corresponding to a medical environment, according to a distance between a source of radiation and a given point in the volume. For example, the propagation processor 422 can identify, according to a linear transformation or a non-linear transformation (e.g., execution of a formula or an equation) a quantity of radiation at a given point in the volume as the distance. For example, the distance between a source of radiation and a given point in the volume can be determined according to a point of a point cloud that defines one or more surfaces within the medical environment according to one or more coordinates that are each indicative of a surface detected at that point. For example, the surface can be detected according to one or more sensors, but is not limited thereto. For example, the object segmenter 416 can obtain point cloud data corresponding to the medical environment and one or more persons or objects therein, or a combination thereof.

In an aspect, the propagation processor 422 can determine a quantity or amount of radiation exposure at a given point in a volume according to a distance between a source of the radiation and a given point in the volume based on one or more electromagnetic models. The point can be part of a surface of an object or a human (e.g., patient, surgeon, medical staff, and so on). For example, the propagation processor 422 can determine the quantity of radiation exposure (e.g., electromagnetic radiation) according to an electric field model (Equation 1), a magnetic field model (Equation 2), or a combination thereof, referred to as electromagnetic wave equation.

$$( {v}_{ph}^{2} {∇}^{2} - \frac{{\partial }^{2}}{\partial {t}^{2}} ) E = 0 \qquad ( Eqn . 1 )$$

$$( {v}_{ph}^{2} {∇}^{2} - \frac{{\partial }^{2}}{\partial {t}^{2}} ) B = 0 \qquad ( Eqn . 2 )$$

For example, vₚₕ corresponds to a phase velocity having a value of the speed of light. Given one or more properties of a radiation source and one or more boundary conditions at a given point in a volume, the propagation processor 422 can determine an intensity field at the given point in the volume. ∇² is the Laplace operator. E stands for electric field, and B stands for magnetic field. For example, properties defining a radiation source can include a location of the radiation source within a volume (e.g., an origin coordinate including one or more spatial positions), an intensity of the radiation source within the volume (e.g., power of radiation in W at the origin coordinate), a direction of the radiation source, and so on. For example, the propagation processor 422 can obtain or determine one or more coordinates that are derived from depth data of one or sensors and according to 3D registration of the sensors to a common coordinate frame, as discussed herein.

For example, the one or more boundary conditions can be defined for elements of a 3D reconstruction of the medical environment including the radiation source. The radiation source can correspond to or defined by a portion (e.g., a point or a set of points) of the radiation system 103 that emit radiation or is configured to emit radiation, as discussed herein. Thus, the propagation processor 422 can, for example, solve one or more of the 3D wave equations (Equation 1) and (Equation 2) for the intensity field originating from the radiation source at a given point in the 3D volume. The propagation processor 422 can account for effects on radiation strength at any given point on a surface identified according to 3D registration of one or more sensors, using the results of (Equation 1) or (Equation 2). Thus, the propagation processor 422 can determine a quantity of radiation exposure at a given point in a volume at a level of accuracy (e.g., high granularity within a volume) and speed (e.g., real-time) beyond the capability of manual processes to achieve.

In an aspect, the radiation data processor 420 can train (e.g., update) a machine learning model to improve accuracy of determination of radiation propagation in a medical environment beyond the capabilities of conventional observation-based processes. For example, the radiation data processor 420 can obtain radiation level data from one or more radiation sensors (e.g., dosimeters) that can collect radiation data as ground truth of radiation levels at various coordinates of the medical environment. For example, each of the dosimeters can be registered to a common coordinate frame, to collect training data corresponding to the ground truth of radiation levels at various positions (defined by sets of coordinates) in the medical environment for a given medical procedure. For example, the dosimeter can measure absorbed radiation at a certain position in the medical environment corresponding to a set of coordinates. For example, the dosimeter can either have a predetermined location or a location detected via object detection or recognition based on the depth data or image data collected from the sensors (e.g., the sensors 140, 150) arranged in the medical environment. With the location of the dosimeter and the dosimeter output radiation, the radiation data processor 420 can determine a prediction of the radiation at the same location according to the machine learning model, and compare the predicted radiation with the dosimeter output radiation to compute a loss according to a loss function to train (e.g., update) the machine learning model. The radiation data processor 420 can execute a training of the machine learning model to minimize the loss (e.g., difference or mean square error (MSE)) between the predicted radiation and ground truth of actual radiation of the dosimeter according to the loss function.

One or more dosimeters can be placed in the medical environment at fixed (predetermined) locations or moveable (dynamic) locations within the medical environment. For example, a fixed location can correspond to a wall, ceiling, object, piece of furniture, or any combination thereof, but is not limited thereto. For example, the predetermined locations can be predefined on a point cloud or 3D reconstruction of the medical environment. For example, the radiation data processor 420 can predict the radiation at the predetermined location in the point cloud or 3D reconstruction, which has a predetermined distance from the radiation system 103. For example, a moveable location can be dynamically identified via machine vision and object detection and identification algorithms based on output data from the sensors (e.g., the sensors 140 and 150). For example, a moveable position can correspond to a position of a wearable radiation sensor worn by a person in the medical environment. The moveable position can be dynamically identified location via machine vision via machine vision and object detection and identification algorithms for the radiation sensor itself, or the moveable position can be approximated using a certain point on a person wearing the radiation sensor.

The coordinate alignment processor 424 can transform or translate one or more coordinate spaces from one or more sensor systems as discussed herein to a radiation system as discussed herein. For example, the coordinate alignment processor 424 can modify or translate a coordinate system according to an origin as discussed herein with respect to the radiation data processor 430. In an aspect, the coordinate alignment processor 424 can align a first coordinate space corresponding to the sensor with a second coordinate space corresponding to the radiation-emitting device. The system can generate the common coordinate space relative to the first location and the second location. In an aspect, the coordinate alignment processor 424 can determine the one or more radiation metrics according to a common coordinate space defining the first location of the sensor within the medical environment relative to the second location of the radiation-emitting device.

The surface characteristic processor 426 can identify one or more types of surfaces of one or more persons or objects as discussed herein. For example, the surface characteristic processor 426 can identify a type of a surface according to one or more of shape, texture, color, or any combination thereof, detected by the first machine learning model with respect to the corresponding portion of the person, object or environment. For example, the surface characteristic processor 426 can identify a protective vest based on a texture of the vest material associated with the medical environment or medical procedure, and can determine that the portion of a surface of the person having the vest has radiation metrics including absorptiveness corresponding to a vest. In an aspect, the surface characteristic processor 426 can determine, by the second machine learning model, a characteristic metric of at least a portion of the object or a portion of the person. In an aspect, the characteristic metric is indicative of at least one of a reflectivity of radiation at the portion of the surface or an absorptiveness of radiation at the portion of the object or the portion of the person.

The radiation event processor 430 can correspond at least partially in one or more of structure and operation to the radiation data processor 340. The radiation event processor 430 can include a radiation metrics processor 432, an environment layout processor 434, and a video annotation processor 436. The radiation metrics processor 432 can link one or more radiation metrics with one or more portions of a surface. For example, the radiation metrics processor 432 can link one or more points of a surface with one or more corresponding radiation metrics. For example, the radiation metrics processor 432 can receive an indication from the surface characteristic processor 426 identifying a portion of a surface as a vest worn by a person and link a first set of radiation metrics with absorptiveness and reflectivity properties corresponding to the vest. For example, the radiation metrics processor 432 can receive an indication from the surface characteristic processor 426 identifying a portion of a surface as a face of a person and link a second set of radiation metrics with absorptiveness and reflectivity properties corresponding to a face. For example, the radiation metrics processor 432 can receive an indication from the surface characteristic processor 426 identifying a portion of a surface as an arm of a person and link a third set of radiation metrics with absorptiveness and reflectivity properties corresponding to an arm. For example, the radiation metrics processor 432 can receive an indication from the surface characteristic processor 426 identifying a portion of a surface of a person as clothed in scrubs and link a fourth set of radiation metrics with absorptiveness and reflectivity properties corresponding to clothing.

The environment layout processor 434 can modify one or more radiation metrics according to a layout of a given medical environment. For example, the environment layout processor 434 can determine whether one or more persons or objects are intervening between the radiation system 103 and a surface as identified and linked with radiation metrics by the radiation metrics processor 432. For example, the environment layout processor 434 can determine one or more surfaces of intervening objects intersecting a vector or path from the radiation system 103 to a given point of a surface or a portion of a surface in the medical environment. The environment layout processor 434 can modify one or more radiation metrics to provide an indication of radiation exposure in view of the intervening objects or person. For example, the environment layout processor 434 can reduce one or more radiation metrics to indicate a lower amount of radiation at a surface of a person or object. For example, the environment layout processor 434 can modify one or more propagation transformations to indicate a lower amount of radiation traveling to a surface of a person or object.

The video annotation processor 436 can generate one or more indications for presentation at a user interface. For example, the video annotation processor 436 can modify a video of a medical environment to indicate radiation exposure for one or more persons or objects in the medical environment. For example, the video annotation processor 436 can generate one or more overlays for one or more persons or objects indicative of a level or quantity of radiation exposure. For example, the video annotation processor 436 can present indications in real time of instantaneous or aggregate radiation exposure for one or more persons or objects in the medical environment (e.g., during a medical procedure). For example, the video annotation processor 436 can present indications in real time to mitigate instantaneous or aggregate radiation exposure for one or more persons or objects in the medical environment (e.g., an indicate to move to another location in the medical environment or to exit the medical environment). The video annotation processor 436 can bidirectionally communicate with the client system 303 to cause the client system 303 to present one or more of the indication in real time or as a recording of a medical procedure or portion thereof. Thus, the computer architecture 400 can provide a plurality of technical solutions according to corresponding technical solutions as discussed herein, but is not limited thereto.

In an aspect, the computer architecture 400 can provide accurate modeling of distribution of scattered radiation in a medical environment, by fusing, according to a 3D volume, features descriptive of X-ray propagation and sensor data corresponding to a given medical environment having a given arrangement and presence of persons and objects. Thus, the first feature of the computer architecture 400 can provide and improve real time motion computation and tracking. For example, as discussed herein, 3D propagation of radiation in and through a medical environment can be computed using data of the radiation system 103 (X-Ray, CBCT) and a 3D reconstruction of a medical environment corresponding to a 3D room layout of the medical environment. Location and composition of various persons and objects can affect distribution of radiation in the medical environment.

In an aspect, the computer architecture 400 can provide real-time guidance system for movement tracking and radiation exposure mitigation for clinicians, beyond the capabilities of conventional observation-based processes. The user interface presentations can provide graphical indications and/or instructions to guide a person to move to or away from certain locations in the medical environment to minimize an amount of radiation exposure to the individual.

In an aspect, the computer architecture 400 can provide real-time guidance for object placement and room layout to minimize radiation exposure. For example, the environment layout processor 434 can iterate through a plurality of object placement configurations, to determine a configuration (e.g., room layout) of persons or objects within the medical environment that minimizes radiation exposure to one or more persons. For example, the environment layout processor 434 can generate radiation metrics for a plurality of persons in a plurality of configurations (e.g., room layouts) of the medical environment, and select the configuration corresponding to a room layout setup that minimizes the amount of radiation exposure to all persons (e.g., patients and clinicians). The real-time guidance for object placement and room layout can combine movement tracking as discussed herein with radiation exposure detection to provide user interface presentations in real-time before and during a medical procedure with guidance for object placement and room layout to minimize radiation exposure.

In an aspect, the computer architecture 400 can provide the clinician radiation exposure (CRE) OPI to provide radiation analytics for post-operative analysis of workflow and pre-operative room layout configurations, as discussed herein. For example, the system enables analysis of workflow in the medical environment and room layout, post-operatively, to educate OR teams, hospital administrators, consultants, and students to better configure the medical and personnel placement to minimize radiation exposure. The radiation metric and the CRE OPI can each be based on a person or object, or on specific body parts of a person based on pose tracking, or an aggregate sum of a plurality of persons or objects. For example, the computer architecture 400 can provide recommendations regarding placement of the radiation system in the medical environment to reduce radiation exposure or CRE OPIs for one or more persons in the medical environment.

For example, the computer architecture 400 can use a human pose estimation model to detect the specific body parts as key points in a human motion model (e.g., a wireframe model) of a body of a person. For example, a set or subset of the key points can correspond to or identify the neck, head, joints, elbows, or other joints or portions of the body of a given person. For example, the computer architecture 400 can use a reference key point such as the head or neck for all persons. The computer architecture 400 can determine average displacement of reference key points of each frame in terms of pixels, using Euclidean distance between pixel coordinates of the reference key point of the person and a reference position (e.g., a coordinate in the medical environment corresponding to (0,0) in a common coordinate frame). For example, the computer architecture 400 can determine distance to objects, person, or surfaces in terms of meters using depth values from a camera perspective. For example, the computer architecture 400 can identify or determine accumulative motion of sterile people and non-sterile people in terms of meter for each period, phase, or task of a workflow of a medical procedure. For example, the computer architecture 400 can use an object shape or positioning estimation model to detect specific devices and equipment in the medical environment via object pose estimation or object detection models. For example, the computer architecture 400 can reduce computational load by modifying a sample rate to determine average a distance value as discussed herein over epochs spanning in the magnitude of seconds (e.g., 1-second epochs).

In an aspect, the computer architecture 400 can provide visualization through at least an extended reality (X R) graphical user interface (GUI). For example, the XR GUI can provide real-time guidance on standing location for a person, an app-based 3D viewer of a computer environment, point cloud colorization based on radiation distribution, and other forms of graphical visual representations.

In an aspect, the computer architecture 400 can provide guidance to clinicians to optimize usage of radiation system 103. For example, the system can optimize the usage of X-ray machines based on real-time calculation of CRE OPI by recommending activation and deactivation at given times during a medical procedure to reduce radiation exposure and to reduce CRE OPIs for one or more persons in the medical environment during the medical procedure. For example, the system can colorize point cloud data based on use case and situation. For example, the surface characteristic processor 426 can colorize point cloud data based on expected of maximum levels of radiation for a given medical procedure or given class of patient in the medical procedure. For example, a user interface can display a 2D representation of point cloud data, radiation metrics or CRE OPIs after a procedure. For example, a user interface can present a body avatar that shows exposure of a person for each body part and that can be colorized or annotated according to the radiation metrics or CRE OPIs.

In an aspect, the radiation event processor 430 can calibrate or modify radiation metrics or models as discussed herein, based on one or more sensors in the medical environment. For example, dosimeters configured to detect radiation levels at given points in the medical environment (e.g., instantaneous or real-time radiation levels) can provide ground truth data indicative of actual radiation levels within the environment. In an aspect, the system can schedule cases and assignment of staff, using the CRE OPI to minimize aggregate radiation exposure over the course of multiple medical procedures.

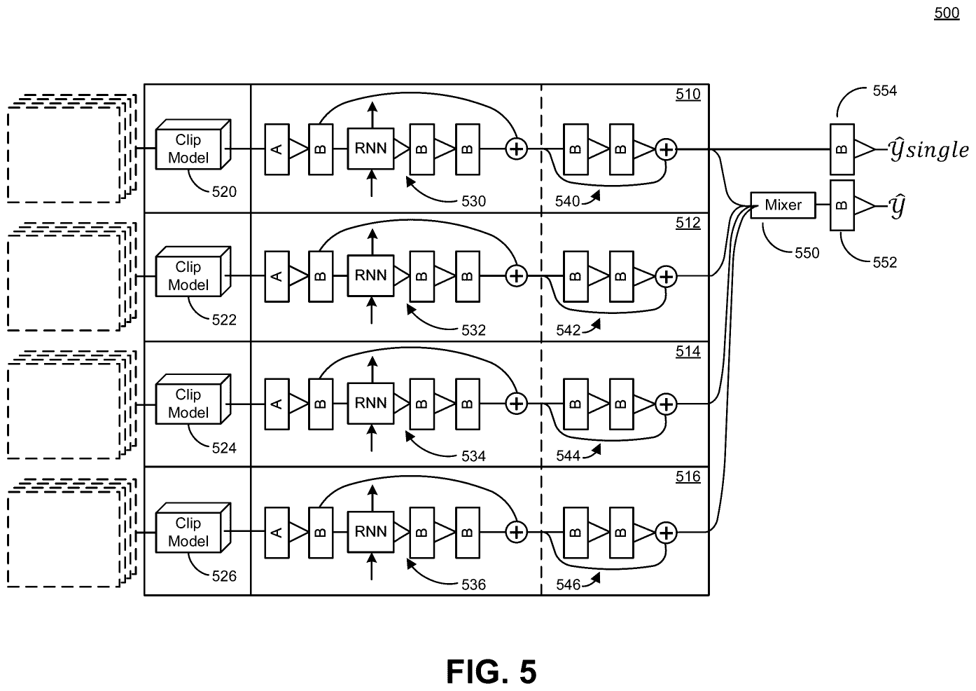

FIG. 5 depicts an example layer model architecture according to this disclosure. As illustrated by way of example in FIG. 5, a layer model architecture 500 can include at least a first layer 510, a second layer 512, a third layer 514, a fourth layer 516, and a mixer 550. For example, the layer model architecture 500 can generate features indicative of one or more body parts or poses of one or more persons, shapes of one or more objects, texture or shapes of one or more items of one or more persons, or any combination thereof, from a first sensor system of the medical environment.

The first layer 510 can correspond to an instance of a vision architecture as discussed herein. For example, the first layer 510 can capture one or more images or video corresponding to a first field of view of a first camera, but is not limited thereto. The first layer 510 can include a first clip model 520, a first layer processor 530, and a first feature processor 540, and can provide output to a layer output 554. The first clip model 520 can include one or more instructions to divide a video into one or more frames, and to select one or more frames corresponding to one or more timestamps or times of capture associated with those one or more frames. The first layer processor 530 can include a first recursive neural network (RNN) to identify one or more image features as input to the first feature processor 540. The first feature processor 540 can generate one or more of the features indicative of one or more body parts or poses of one or more persons, shapes of one or more objects, texture or shapes of one or more items of one or more persons, or any combination thereof, from a first sensor system of the medical environment. The first layer processor 530 can be coupled with one or more processing devices at inputs and outputs thereof. For example, the processing devices can have different memory capacities, including as illustrated in FIG. 5 by way of example, a first memory size A and a second memory size B. For example, a first memory size “A” can correspond to a memory capacity of 1024 bits or bytes, and a second memory size “B” can correspond to a memory capacity of 128 bits or bytes. However, this disclosure is not limited to the memory sizes or the particular configuration of memory sizes illustrated herein by way of example. For example, the first layer processor 530 can have an input coupled with a first memory device of size A in series with a second memory device of size B, and can have an output coupled with a first memory device of size B in series with a second memory device also of size B.

The second layer 512 can correspond to an instance of a vision architecture as discussed herein. For example, the second layer 512 can capture one or more images or video corresponding to a second field of view corresponding to a second camera, but is not limited thereto. The second layer 512 can include a second clip model 522, a second layer processor 532, and a second feature processor 542. The second layer processor 532 can include a second RNN to identify one or more image features as input to the second feature processor 542. The second feature processor 542 can generate one or more of the features indicative of one or more body parts or poses of one or more persons, shapes of one or more objects, texture or shapes of one or more items of one or more persons, or any combination thereof, from a second sensor system of the medical environment.

The third layer 514 can correspond to an instance of a vision architecture as discussed herein. For example, the third layer 514 can capture one or more images or video corresponding to a third field of view corresponding to a third camera, but is not limited thereto. The third layer 514 can include a third clip model 524, a third layer processor 534, and a third feature processor 544. The third layer processor 534 can include a third RNN to identify one or more image features as input to the third feature processor 544. The third feature processor 544 can generate one or more of the features indicative of one or more body parts or poses of one or more persons, shapes of one or more objects, texture or shapes of one or more items of one or more persons, or any combination thereof, from a third sensor system of the medical environment.

The fourth layer 516 can correspond to an instance of a vision architecture as discussed herein. For example, the fourth layer 516 can capture one or more images or video corresponding to a fourth field of view of a fourth camera, but is not limited thereto. The fourth layer 516 can include a fourth clip model 526, a fourth layer processor 536, and a fourth feature processor 546. The fourth layer processor 536 can include a fourth RNN to identify one or more image features as input to the fourth feature processor 546. The fourth feature processor 546 can generate one or more of the features indicative of one or more body parts or poses of one or more persons, shapes of one or more objects, texture or shapes of one or more items of one or more persons, or any combination thereof, from a fourth sensor system of the medical environment. This disclosure is not limited to the layers discussed herein.

The mixer 550 can aggregate output from each of the first, second, third, and fourth layers 510, 512, 514 and 516. Thus, the mixer 550 can provide a fused output 552 based on predictions output by each of the first, second, third, and fourth layers 510, 512, 514 and 516. The layer output 554 can correspond to an output of the first layer 510. For example, the layer output 554 can correspond to a prediction output by the first layer 530. The layer output 554 is not limited to the example illustrated herein. For example, one or more of the second, third and fourth layers 512, 514 and 516 can provide layer outputs that correspond at least partially in one or more of structure and operation to the layer output 554.

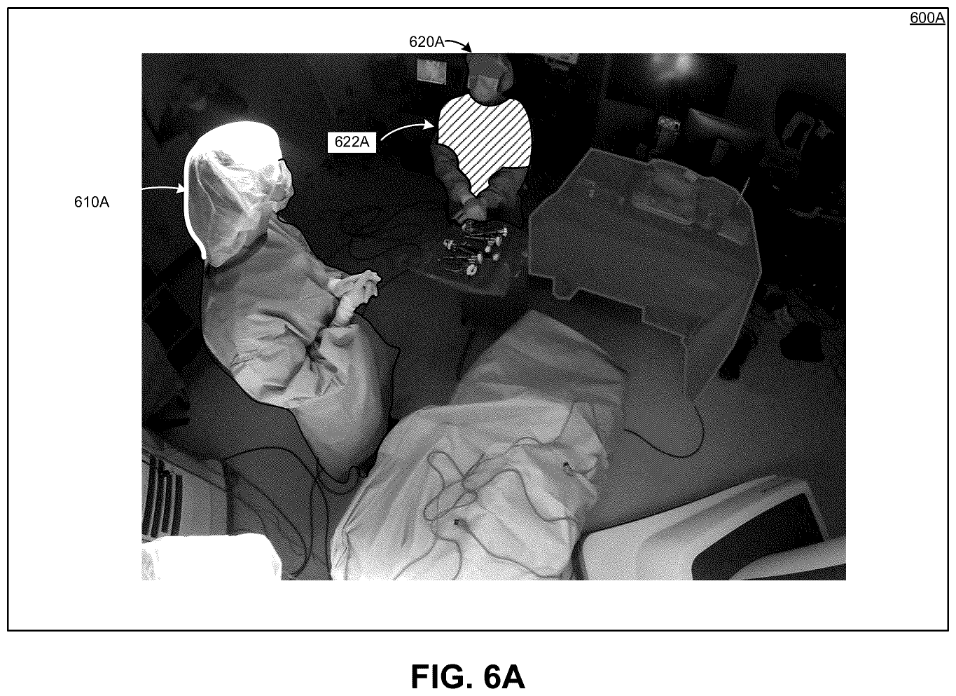

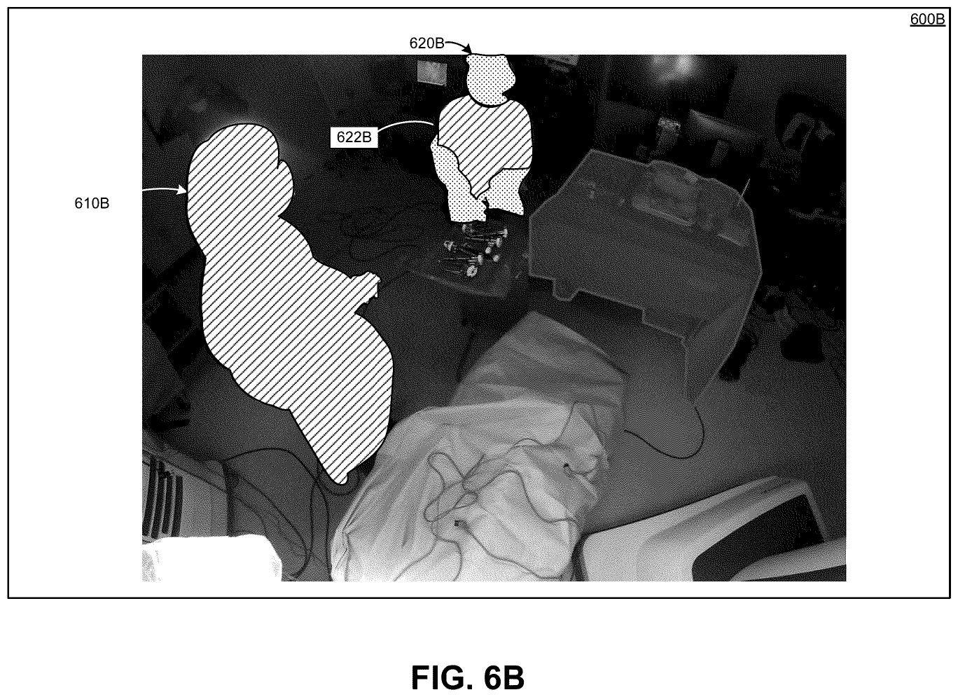

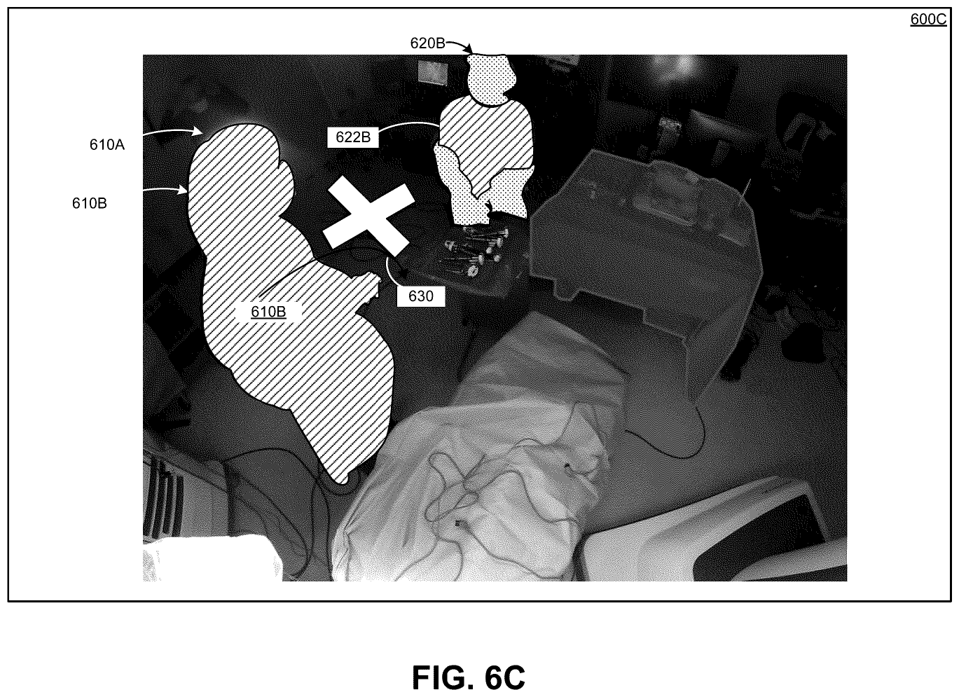

FIG. 6A depicts an example detection of radiation surface characteristics, according to this disclosure. As illustrated by way of example in FIG. 6A, a detection of radiation surface characteristics 600A can include at least a first person 610A, and a second person 620A. The first person 610A can be located in the medical environment at a first position with respect to a radiation system 103. The first person 610A can be wearing medical scrubs, without a radiation vest to mitigate radiation exposure of the first person 610A. As discussed herein, a CRE OPI can be derived from a metric indicative of radiation exposure of a person at a given time or location. For example, the CRE OPI can be determined as an aggregate radiation exposure of a person over time or over the course of a medical procedure. The second person 620A can be located in the medical environment at a second position with respect to a radiation system 103. For example, the second position can be farther from the radiation system 103 than the first position. The second person 620A can be wearing medical scrubs, and can be wearing a radiation vest to mitigate radiation exposure of the second person 620A. The second person 620A can have a portion corresponding to a radiation absorption surface 622A that is aligned with the radiation vest. The radiation absorption surface 622A can have radiation metrics distinct from radiation metrics of the second person 620A external to the radiation absorption surface 622A (e.g., face, hands, scrubs). For example, the radiation metrics of the radiation absorption surface 622A can indicate higher radiation absorptiveness than radiation metrics of the surface of the second person 620A external to the radiation absorption surface 622A.

FIG. 6B depicts an example detection of radiation exposure, according to this disclosure. As illustrated by way of example in FIG. 6B, a detection of radiation exposure 600B can include at least a complete exposure indication 610B for the first person 610A, and a first partial exposure indication 620B for the exposed portion of the second person 620A. The complete exposure indication 610B for the first person 610A can indicate radiation exposure aggregated over the surface corresponding to the first person 610A. For example, the complete exposure indication 610B can correspond to an overlay having a partially transparent red color corresponding to a “mitigate now” level. The first partial exposure indication 620B for the exposed portion of the second person 620A can indicate radiation exposure aggregated over the surface corresponding to the second person 620A external to the radiation absorption surface 622A (e.g., face, hands, scrubs). For example, the first partial exposure indication 620B can correspond to an overlay having a partially transparent yellow color corresponding to a “within limit” level. The first partial exposure indication 620B for the exposed portion of the second person 620A can include a second partial exposure indication 622B for the unexposed portion of the second person 620A. The second partial exposure indication 622B for the unexposed portion of the second person 620A can indicate radiation exposure aggregated over the surface corresponding to the radiation absorption surface 622A (e.g., protective vest). For example, the second partial exposure indication 622B can correspond to an overlay having a partially transparent green color corresponding to a “mitigated” level.

In an aspect, the client system 303 can present, via the user interface 360, a visual indication of the second output at a portion of the one or more images corresponding to the person or the object. In an aspect, the visual indication corresponds to an overlay at the portion of the one or more images corresponding to the person or the object. In an aspect, the visual indication has at least one of a color or an opacity indicative of the quantity of radiation exposure of the object or the person at the first location. In an aspect, the visual indication corresponds to the quantity of radiation exposure of the object or the person.

FIG. 6C depicts an example mitigation of radiation exposure, according to this disclosure. As illustrated by way of example in FIG. 6C, a mitigation of radiation exposure 600C can include at least a positional indication 630 for the first person 610A. The positional indication 630 for the first person 610A can indicate a position identified by the environment layout processor 434 to reduce radiation exposure of the first person 610A. For example, the video annotation processor 436 can cause the user interface 360 to present the positional indication 630 in response to a determination that first person 610A is associated with a “mitigate now” level. Thus, the environment layout processor 434 can provide a technical improvement at least to provide real-time feedback to persons in a medical environment to mitigate radiation exposure at an individualized level of granularity and with real-time responsiveness beyond the capability of manual processes.

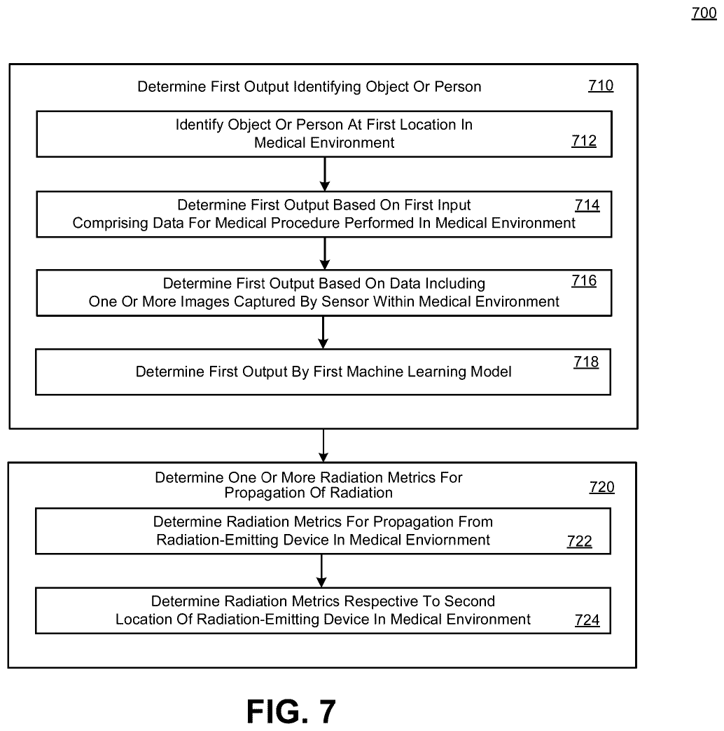

FIG. 7 depicts an example method of detection and mitigation of radiation exposure in medical environments, according to this disclosure. At least one of the system 300, the data processing system 302, or any combination thereof, can perform method 700. In an aspect, the method 700 can include aligning a first coordinate space corresponding to the sensor with a second coordinate space corresponding to the radiation-emitting device. The method can include generating the common coordinate space relative to the first location and the second location.

At 710, the method 700 can determine a first output identifying an object or a person. At 712, the method 700 can identify the object or the person at a first location in the medical environment. In an aspect, the method can include generating, by the first machine learning model, the first location in real time during the medical procedure. At 714, the method 700 can determine the first output based on a first input comprising data for a medical procedure performed in the medical environment. At 716, the method 700 can determine the first output based on data including one or more images captured by a sensor within the medical environment. At 718, the method 700 can determine the first output by a first machine learning model.

At 720, the method 700 can determine one or more radiation metrics for propagation of radiation. In an aspect, the method can include determining the one or more radiation metrics according to a common coordinate space defining the first location of the sensor within the medical environment relative to the second location of the radiation-emitting device. At 722, the method 700 can determine the radiation metrics for propagation from the radiation-emitting device through the medical environment. At 724, the method 700 can determine the radiation metrics respective to a second location of a radiation-emitting device in a medical environment. In an aspect, the method can include determining, by the second machine learning model, a characteristic metric of at least a portion of the object or a portion of the person.

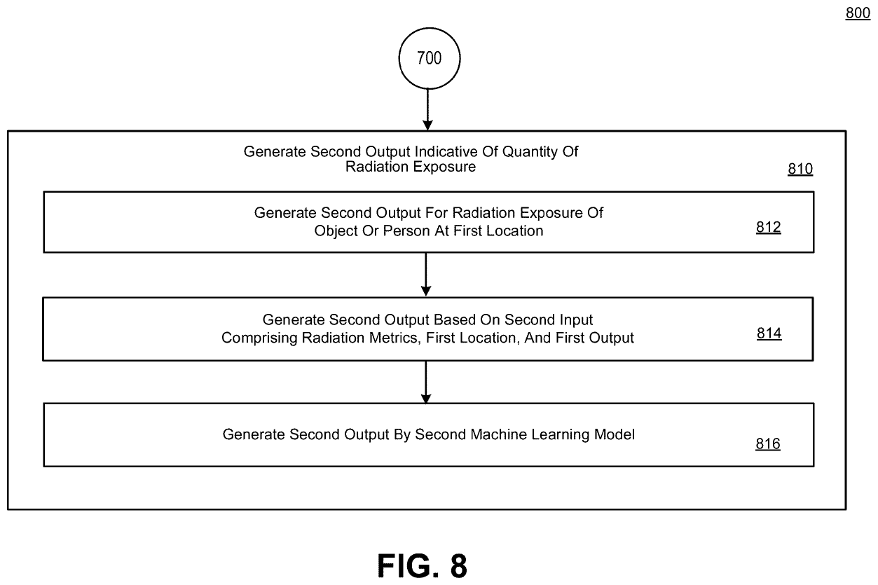

FIG. 8 depicts an example method of detection and mitigation of radiation exposure in medical environments, according to this disclosure. At least the system 300, the data processing system 302, or any combination thereof, can perform method 800. At 810, the method 800 can generate a second output indicative of a quantity of radiation exposure. At 812, the method 800 can generate the second output for radiation exposure of the object or the person at the first location. At 814, the method 800 can generate the second output based on a second input comprising the radiation metrics, the first location, and the first output. At 816, the method 800 can generate the second output by a second machine learning model.

In an aspect, the method 800 can include presenting, via the user interface 360, a visual indication of the second output at a portion of the one or more images corresponding to the person or the object. In an aspect, the visual indication corresponds to an overlay at the portion of the one or more images corresponding to the person or the object. In an aspect, the visual indication has at least one of a color or an opacity indicative of the quantity of radiation exposure of the object or the person at the first location. In an aspect, the non-transitory computer readable medium can include one or more instructions executable by the system processor 310. The system processor 310 can cause, the user interface 360 to present a visual indication of the second output at a portion of the one or more images corresponding to the person or the object.

Having now described some illustrative implementations, the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items. References to “is” or “are” may be construed as nonlimiting to the implementation or action referenced in connection with that term. The terms “is” or “are” or any tense or derivative thereof, are interchangeable and synonymous with “can be” as used herein, unless stated otherwise herein.

Directional indicators depicted herein are example directions to facilitate understanding of the examples discussed herein, and are not limited to the directional indicators depicted herein. Any directional indicator depicted herein can be modified to the reverse direction, or can be modified to include both the depicted direction and a direction reverse to the depicted direction, unless stated otherwise herein. While operations are depicted in the drawings in a particular order, such operations are not required to be performed in the particular order shown or in sequential order, and all illustrated operations are not required to be performed. Actions described herein can be performed in a different order. Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Scope of the systems and methods described herein is thus indicated by the appended claims, rather than the foregoing description. The scope of the claims includes equivalents to the meaning and scope of the appended claims.

What is claimed is:

1. A system, comprising:
one or more processors, coupled with memory, to:
determine, by a first machine learning model based on a first input comprising data for a medical procedure performed in a medical environment, a first output identifying an object or a person at a first location in the medical environment, the data including one or more images captured by a sensor within the medical environment;
determine, respective to a second location of a radiation-emitting device in a medical environment, one or more radiation metrics corresponding to propagation of radiation from the radiation-emitting device through the medical environment; and
generate, by a second machine learning model based on a second input comprising the radiation metrics, the first location, and the first output, a second output indicative of a quantity of radiation exposure of the object or the person at the first location.

2. The system of claim 1, the processors to:
generate, by the first machine learning model, the first location in real time during the medical procedure.

3. The system of claim 1, the processors to:
determine the one or more radiation metrics according to a common coordinate space defining the first location of the sensor within the medical environment relative to the second location of the radiation-emitting device.

4. The system of claim 3, the processors to:
align a first coordinate space corresponding to the sensor with a second coordinate space corresponding to the radiation-emitting device; and
generate the common coordinate space relative to the first location and the second location.

5. The system of claim 1, the processors to:
determine, by the second machine learning model, a characteristic metric of at least a portion of the object or a portion of the person.

6. The system of claim 5, wherein the characteristic metric is indicative of at least one of a reflectivity of radiation at the portion of a surface of the object or the person at the first location or an absorptiveness of radiation at the portion of the surface.

7. The system of claim 5, the processors to:
identify, by the first machine learning model, at least one item associated with the portion of the object or the portion of the person; and
determine, by the second machine learning model based on the one or more items, the characteristic metric based on an association of the portion of the object or the portion of the person with the item.

8. The system of claim 5, wherein the portion of the object or the portion of the person corresponds to a portion of a point cloud associated with the object or the person.

9. The system of claim 1, the processors to:
present, via a user interface, a visual indication of the second output at a portion of the one or more images corresponding to the person or the object.

10. The system of claim 9, wherein the visual indication corresponds to an overlay at the portion of the one or more images corresponding to the person or the object.

11. The system of claim 9, wherein the visual indication has at least one of a color or an opacity indicative of the quantity of radiation exposure of the object or the person at the first location.

12. The system of claim 9, wherein the visual indication corresponds to the quantity of radiation exposure of the object or the person.

13. A method, comprising:
determining, by a first machine learning model based on a first input comprising data for a medical procedure performed in a medical environment, a first output identifying an object or a person at a first location in the medical environment, the data including one or more images captured by a sensor within the medical environment;
determining, respective to a second location of a radiation-emitting device in a medical environment, one or more radiation metrics corresponding to propagation of radiation from the radiation-emitting device through the medical environment; and
generating, by a second machine learning model based on a second input comprising the radiation metrics, the first location, and the first output, a second output indicative of a quantity of radiation exposure of the object or the person at the first location.

14. The method of claim 13, further comprising:
generating, by the first machine learning model, the first location in real time during the medical procedure.

15. The method of claim 13, further comprising:
determining the one or more radiation metrics according to a common coordinate space defining the first location of the sensor within the medical environment relative to the second location of the radiation-emitting device.

14. The method of claim 15, further comprising:
aligning a first coordinate space corresponding to the sensor with a second coordinate space corresponding to the radiation-emitting device; and
generating the common coordinate space relative to the first location and the second location.

15. The method of claim 13, further comprising:
determining, by the second machine learning model, a characteristic metric of at least a portion of the object or a portion of the person.

16. The method of claim 13, further comprising:
presenting, via a user interface, a visual indication of the second output at a portion of the one or more images corresponding to the person or the object.

17. The method of claim 16, wherein the visual indication corresponds to an overlay at the portion of the one or more images corresponding to the person or the object.

18. The method of claim 16, wherein the visual indication has at least one of a color or an opacity indicative of the quantity of radiation exposure of the object or the person at the first location.

19. A non-transitory computer readable medium including one or more instructions stored thereon and executable by a processor to:
determine, by the processor via a first machine learning model based on a first input comprising data for a medical procedure performed in a medical environment, a first output identifying an object or a person at a first location in the medical environment, the data including one or more images captured by a sensor within the medical environment;
determine, the processor and respective to a second location of a radiation-emitting device in a medical environment, one or more radiation metrics corresponding to propagation of radiation from the radiation-emitting device through the medical environment; and
generate, by the processor via a second machine learning model based on a second input comprising the radiation metrics, the first location, and the first output, a second output indicative of a quantity of radiation exposure of the object or the person at the first location.

20. The non-transitory computer readable medium of claim 19, the non-transitory computer readable medium further including one or more instructions executable by the processor to:
cause, by the processor, a user interface to present a visual indication of the second output at a portion of the one or more images corresponding to the person or the object.
